(19) United States
(12) Patent Application Publication
(10) Pub. No.: US 2005/0241648 A1
(43) Pub. Date: Nov. 3, 2005

(54) Patient privacy screen

(76) Inventor: Michael Dobbs, Pebble Beach, CA (US)

Correspondence Address: US CORPORATIONS, INC., 630 NEPTUNE AVE., ENCINITAS, CA, 92024, US

(21) Appl. No.: 11/075,590

(22) Filed: Mar. 8, 2005

Related U.S. Application Data
(60) Provisional application No. 60/567,001, filed on Apr. 30, 2004.

(51) Int. Cl. A61F 11/00

(52) U.S. Cl. 128/857

(57) ABSTRACT

A patient privacy screen device to be used in conjunction with a hospital or ambulance gurney while a patient is being cared for or transported from one place to another. The device allows quick and effortless erection of two solid side curtains and one solid head by the medical attendees.

### CROSS-REFERENCE TO RELATED APPLICATION

This application relies on a previously filed provisional application 60/567001 043004

### STATEMENT REGARDING FEDERALLY SPONSORED RESEARCH OR DEVELOPMENT

Not applicable

### REFERENCE TO SEQUENCE LISTING

Not applicable

### BACKGROUND OF THE INVENTION

This invention relates to privacy on patient transport systems, and more particularly, to a patient transport system for transferring an immobile patient from one location to another.

The current device is a self contained, self supporting, portable, collapsible device to provide privacy for patients who are on gurneys while in public areas

The current device is slid under the mattress of a gurney or patient bed in a hospital or other healthcare facility. Then the collapsible screen can be extended from above the reclining patient's head down to their waist to provide privacy while in public areas or while being transported from one place to another

The invention is also usable in hospitals and elsewhere to move an individual such as a patient from a hospital gurney or EMS stretcher to a hospital bed

### SUMMARY OF THE INVENTION

It is therefore an object of the invention to provide a contained, self supporting, portable, collapsible device to provide privacy for patients for use in connection with a stretcher and/or gurney used to transport an invalid or an injured individual from an outside area of the hospital to the hospital or from one part of the hospital to another part of the hospital.

Another object of the invention is to provide for a contained, self supporting, portable, collapsible device to provide privacy for patients to the ambulance/paramedic stretcher pad and the hospital emergency room stretcher/gurney pad.

For this purpose, the present invention proposes to provide for a contained, self supporting, portable, collapsible device to provide privacy for patients having a substantially oblong dimension in which the length is substantially longer than the width. Since a mattress for use with an EMS stretcher or a hospital stretcher or gurney is generally of a width of about 24 and 30 inches, respectively, and a length of about 72 and 76 inches, respectively, a contained, self supporting, portable, collapsible device to provide privacy for patients for the stretcher is desirably provided privacy at each of the sides of the stretcher or gurney.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and. scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a new Patient Privacy Screen which has many of the advantages of the closures mentioned heretofore and many novel features that result in a patient privacy screen device which is not anticipated, rendered obvious, suggested, or even implied by any of the prior art tool guides, either alone or in any combination thereof.

It is another object of the present invention to provide a patient privacy screen device which may be easily and efficiently manufactured and marketed.

It is a further object of the present invention to provide a patient privacy screen device which is of a durable and reliable construction.

An even further object of the present invention is to provide a patient privacy screen device which is susceptible of a low cost of manufacture with regard to both materials and labor, and which accordingly is then susceptible of low prices of sale to the consuming public, thereby making such patient privacy screen device economically available to the buying public.

Still yet another object of the present invention is to provide a patient privacy screen device which provides in the apparatuses and methods of the prior art some of the advantages thereof, while simultaneously overcoming some of the disadvantages normally associated therewith.

Yet another object of the present invention is to provide a new patient privacy screen device which includes a collapsible and extendable frame of weather resistant material.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

These and other features of the invention will be more fully appreciated from the following detailed descriptions, taken in conjunction with the accompanying drawings, in which like parts are given like reference numerals and wherein:

FIG. 1 is a schematic perspective right side view of the collapsed patient privacy screen as mounted on a gurney.

FIG. 2 1 is a schematic perspective right side view of the patient privacy screen device in use as mounted on a gurney.

### DETAILED DESCRIPTION

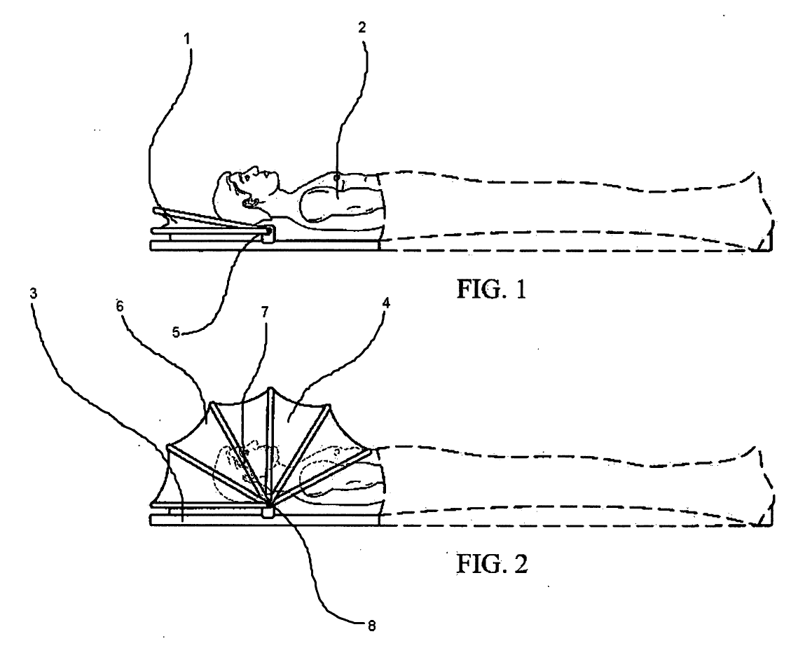

FIGS. 1-2 illustrate embodiments of the privacy screen device according to the principles of the invention.

FIG. 1 as seen, the patient privacy screen 1, mounts on the upper or top side of a hospital or ambulance gurney which supports a patient 2. The device maintains a collapsible hinge 5 allowing the screen 1 to be reduced in size and free the medical staff to care for the patient.

FIG. 2 illustrates the current apparatus in use according to the principles of the invention. The screen device 6, is mounted on the upper or top side of any hospital or ambulance gurney 3, allowing the patient to be safely covered while in transit or while being cared for in a public place. The screen material 4 is of a collapsible, light weight cloth allowing the structural members 7, to easily support and control the material. The screen device operates on hinge 8, allowing it to be collapsed when not in use.

As illustrated, in FIGS. 1-3 will be described in detail on the appended illustration submittal.

1. A portable, attachable patient privacy screen device for hospital and ambulance gurneys and the like where patients are transported from one location to another under medical supervision.

2. A patient privacy screen device according to claim 1 wherein the device is placed under the gurney mattress.

3. A patient privacy screen device according to claim 1 wherein the device is retractable in such a manor where the sides and top of the device are concealed until use.

4. A patient privacy screen device according to claim 1 wherein the device provides for patient privacy from the head to mid body area.

5. A patient privacy screen device according to claim 1 wherein the device body is maintained under the gurney mattress even when the sides and top have been extended and erected for use.

6. A patient privacy screen device according to claim 1 wherein the device provides privacy for the transported patient from public or private vision during medical care.

7. A patient privacy screen device according to claim 1 wherein the device maintains solid privacy curtains which are retractable under the patient and gurney mattress
